(19) United States
(12) Patent Application Publication
(10) Pub. No.: US 2014/0186473 A1
(43) Pub. Date: Jul. 3, 2014

(54) COMPOSITION FOR TREATING OBESITY AND DIABETES AND FOR INCREASING MUSCLE MASS AND IMPROVING CAPACITY FOR EXERCISE, COMPRISING EXTRACTS OF PIPER RETROFRACTUM VAHL. FRUITS AS ACTIVE INGREDIENTS

(71) Applicants: Kyung-Jin Kim, Seoul (KR); Min-Sun Kim, Seoul (KR); Jae-Kwan Hwang, Goyang-si (KR)

(72) Inventors: Kyung-Jin Kim, Seoul (KR); Min-Sun Kim, Seoul (KR); Jae-Kwan Hwang, Goyang-si (KR)

(73) Assignees: INDUSTRY-ACADEMIC COOPERATION FOUNDATION YONSEI UNIVERSITY, Seoul (KR); CJ CHEILJEDANG CORP., Seoul (KR)

(21) Appl. No.: 14/122,752

(22) PCT Filed: May 31, 2012
(86) PCT No.: PCT/KR2012/004325

§ 371 Date: Nov. 27, 2013

(30) Foreign Application Priority Data
May 31, 2011 (KR) 10-2011-0052190
May 31, 2011 (KR) 10-2011-0052191
Jun. 2, 2011 (KR) 10-2011-0053484

(51) Int. Cl. A61K 36/67; A23L 1/30

(52) U.S. Cl. CPC A61K 36/67; A23L 1/3002
USPC 424/734

(57) ABSTRACT

The present invention relates to a food composition, and to a pharmaceutical composition, which exhibit anti-obesity effects by means of a thermogenesis produced by an activity of a UCP of fat cells, which exhibit anti-diabetes effects by means of an improved insulin resistance, increase muscle mass, and improve capacity for exercise. More particularly, the present invention relates to a novel use of a composition containing extracts of piper retrofractum, and to the food composition or pharmaceutical composition which exhibit anti-obesity effects by means of decreased body weight and body fat, exhibit anti-diabetes effects by means of ameliorated insulin resistance through the reduction of blood glucose and blood insulin, increase muscle mass by increasing an energy source being supplied to the muscles, and improve the capacity for exercise including increased endurance.

### TECHNICAL FIELD

The present invention relates to a composition for treating obesity and diabetes, for increasing muscle mass, and for enhancing exercise performance. More particularly, the present invention relates to the novel use of a composition comprising an extract from Piper retrofractum Vahl. in inducing thermogenesis so as to lose weight and body fat, to decrease blood glucose and insulin levels, and to increase the supply of energy to muscle, and therefore, the present invention provides a functional food or pharmaceutical composition for preventing and treating obesity or diabetes, or for enhancing exercise performance including endurance.

### BACKGROUND ART

According to the 2010 OECD report on obesity, about 3% of the adult population in Korea is obese (BMI 30 or higher), with 30% (BMI 25˜30) found in 30% of the population. In the world, there are as many as 250 million obese people and this number is expected to expand to 300 million by 2025. For obesity treatment, body calories must be consumed by increasing energy consumption, relative to energy uptake. Among approaches to consuming energy in the body are physical exercise and thermogenesis. Regular exercise with certain intensity is required, and extensive research has been conducted into the activation of exercise-related genes so as to promote the efficiency of physical exercise. Thermogenesis is the production of heat production in organisms in which, for example, UCPs (uncoupling proteins), a kind of mitochondrial transport protein, are induced to increase protein leak, generating heat through uncoupling oxidation, without ATP production. Of UCPs, which function to control energy homeostasis in the body, UCP 1 is found in BAT (brown adipose tissue), while UCP 2 and UCP 3 are present in muscles and adipose tissues. In addition, obese persons are different from non-obese persons in body metabolisms including fat and glucose metabolisms, as a consequence of insulin dysfunction such as due to insulin resistance. Persistent insulin resistance finally leads to diabetes, and in fact, a great number of diabetes cases are reportedly attributed to obesity.

Reductil and Xenical, which are representative of currently used anti-obesity drugs, are reported to produce various side effects including thirst, dizziness, a sense of abdominal distension, and steatorrhea. In recent years, studies have actively been focused on the development of functional foods based on natural substances, safe and almost free of side effects, rather than synthetic chemicals.

Extracts from Piper retrofractum Vahl., which is a medicinal herb used traditionally in southeastern Asia, were found to cause the loss of weight and body fat in obesity-induced animals after long-term uptake of the extracts, which is thought to result from thermogenesis based on UCP activation in adipose cells.

Anti-obesity herb extracts are disclosed in Korean Patent Application No. 10-2001-22510 for a puer tea composition, in Korean Patent Application No. 10-2003-87280 for Cucurbita sp. in Korean Patent Application No. 10-2006-115006 for red ginseng mixture, in Korean Patent Application No. 10-2007-56192 for Lysimachiae Foenum-Graeci Herba, in Korean Patent Application No. 10-2006-38254 for Pleurospermum kamtschaticu extract, in Korean Patent Application No. 10-2007-82438 for a Vitis vinifera extract, and in Korean Patent Application No. 10-2006-91698 for a Cassia tora Linne extract.

Diabetes is classified into type 1 and type 2 varieties. Type 2 diabetes, also called adult-onset diabetes, is characterized by insulin resistance, which may be combined with relatively reduced insulin secretion. The defective responsiveness of body tissues to insulin involves various factors including a reduction in insulin receptors, the intracellular depeletion of IRS-1 (insulin receptor substrate-1), and poor tyrosine kinase activity. This disease is chronic, and leads to various metabolic abnormalities, with the subsequent development of various complications such as diabetic retinopathy, renal failure, diabetic foot, etc. High incidences of diabetes are reported in advanced countries.

TZD drugs, such as troglitazone, pioglitazoe and rogigaltazone, although less apt to cause side effects, compared to other diabetes therapeutics, still remain causative of hepatotoxicity and obesity.

Rather than such synthetic chemicals, plant extracts which are safe and fairly free of side effects, have recently been studied for availability for functional foods.

With regard to anti-obesity effects, an Inonotus obliquus extract is described in Korean Patent Application No. 10-2005-36879, an Alnus japonica extract in Korean Patent Application No. 10-2002-48813, a Fomes Fomantarius extract in Korean Patent Application No. 10-2003-69748, and a mulberry leaf extract in Korean Patent Application No. 10-2004-82926.

Overweight or obese persons, or normal persons but with high body fat can prevent and escape from metabolic syndromes such as diabetes, hypertension, etc., by increasing muscle mass while decreasing body fat. In addition, an increase in muscle mass is accompanied by an increase of basal metabolism, thus allowing for an effective workout diet without a yo-yo phenomenon. To increase muscle mass, exercise, diet therapy, and ergogenic aid may be used. Intensive studies on functional foods for enhancing performance in exercise have been actively conducted in Eastern and Western countries. Given a certain level, chemical compounds, such as steroids, caffeine, sodium hydrogen carbonate, and sodium citrate, can significantly enhance exercise performance, but evoke significant side effects which may be fatal.

More particularly, blood free fatty acids (FFA), and intramuscular triglyceride (IMTG) translocate into mitochondria where they are converted into energy for muscle. In this regard, carnitine palmitoyltransferase-1 (CPT-1) plays an important role. The enzyme is known as to function as an important regulator in the transfer of long-chain fatty acids to the mitochondria wherein the β-oxidation is performed.

Acetyl-CoA carboxylase (ACC) is inactivated through phosphorylation by 5′ AMP-activated protein kinase (AMPK), which performs a mechanical role of sensing cellular energy levels. A bout of exercise increases the activity of AMPK, leading to phosphorylating and deactivating ACC, with the consequent reduction of malonyl-CoA. A reduced level of malonyl-CoA invokes the activation of CPT-1, which, in turn, increases the influx of acylated fatty acids into the mitochondria.

Recently, plant extracts, which are safe and fairly free of side effects, rather than synthetic chemicals, have actively been studied for availability for functional foods.

An agent for enhancing muscle mass is described in Korean Patent Application No. 10-2005-709019 while a food composition for enhancing performance in exercise is described in Korean Patent Application No. 10-2003-38486. Korean Patent Application Nos. 10-1997-12503 and 10-2007-104898 addresses an anabolic ginseng product and a dietary food composition for enhancing exercise performance, respectively.

Piper retrofractum Vahl., a plant belonging to the piperaceae family, grows naturally in the southeastern Asia, and its fruits are used in the present invention.

This plant has traditionally been used as a spice, a seasoning, a therapeutic for abdominal and intestinal diseases, and a thermogen for maintaining body temperature against cold weather. Piper retrofractum Vahl. is found to contain alkamide constituents including retrofractamide A, retrofractamide B (pipericide), retrofractamide C, piperine, guineensine, methyl piperate, pelliorine, and piperlongumininie, which are known for various activities such as antioxidation, antibacterial and pesticidal activity, improving drug bioavailability, and for use in vasodilatation-induced thermogenesis.

Nowhere in previous literature has Piper retrofractum Vahl. been reported for its activities including the weight and body fat loss caused by UCP activation-induced thermogenesis, the prophylaxis and therapy of diabetes by decreasing fasting glucose levels and blood insulin levels, and the increase of muscle mass and exercise performance by ACC inhibition and CPT-1 activation driven by AMPK activity.

In the present invention, fruits of Piper retrofractum Vahl., a medicinal plant of the southeastern Asia origin, is found to significantly decrease body weight and fat in obesity-induced animals after the long-term uptake thereof, as well as to reduce blood glucose and blood insulin levels in these animals, thus exhibiting an anti-diabetic effect. In this context, experimental animals were induced to fall into obese and diabetic states by being fed a high-fat diet for 7 weeks thereto, and then followed by Piper retrofractum Vahl. for 8 weeks while monitoring them for reduction in weight and body fat as well as blood glucose and insulin levels. Effective decreases in both the parameters were detected, indicating that the extracts can be used as therapeutics for the prevention and treatment of obesity and diabetes, or as anti-obesity and anti-diabetic functional food compositions or pharmaceutical preparations.

### DISCLOSURE

### Technical Problem

It is therefore an object of the present invention to provide a composition comprising a Piper retrofractum Vahl. fruit extract as an active ingredient, and a pharmaceutical composition comprising the same.

### Technical Solution

The object of the present invention may be achieved by obtaining an extract from Piper retrofractum Vahl. fruits, followed by in vivo experiments in which the extract is administered to model mice with obesity and diabetes, and evaluated for anti-obesity and anti-diabetic effects and for the enhancement of muscle mass and exercise performance.

### Advantageous Effects

A composition comprising a Piper retrofractum Vahl. extract in accordance with the present invention can prevent and treat obesity by decreasing body weight and fat, and can prevent diabetes by reducing blood glucose and insulin levels while showing a significant improvement in muscle mass and exercise performance.

### DESCRIPTION OF DRAWINGS

FIG. 1 is a graph showing the effect of the Piper retrofractum Vahl. extract according to the present invention on body weight loss in mice. The marks * and ** on the graph represent significance at *p<0.05 and **p<0.01), respectively, as compared by one-way ANOVA and Tukey's Multiple Comparison Test.

FIG. 2 is a graph showing a reduction in body fat upon the treatment of mice with the Piper retrofractum Vahl. Extract of the present invention. The marks * and ** on the bar graph represent significance at *p<0.05 and **p<0.01), respectively, as compared by one-way ANOVA and Tukey's Multiple Comparison Test.

FIG. 3 shows the activation of UCP3 protein in mouse adipose cells (3T3-L1) treated with the Piper retrofractum Vahl. extract of the present invention, as analyzed by Western blotting.

FIG. 4 is a graph showing the effect of the Piper retrofractum Vahl. extract of the present invention on fasting glucose level in mice. The marks * and ** on the bar graph represent significance at *p<0.05 and **p<0.01), respectively, as compared by one-way ANOVA and Tukey's Multiple Comparison Test.

FIG. 5 is a graph showing the effect of the Piper retrofractum Vahl. extract of the present invention on fasting insulin level in mice. The marks and * and ** on the bar graph represent significance at *p<0.05 and **p<0.01), respectively, as compared by one-way ANOVA and Tukey's Multiple Comparison Test.

FIG. 6 is a graph showing a reduction of HOMA-IR in mice treated with the Piper retrofractum Vahl. extract of the present invention.

FIG. 7 shows the activation of IRS-1 in mouse livers treated with the Piper retrofractum Vahl. extract of the present invention, as analyzed by Western blotting.

FIG. 8 shows PET/CT scan images of thigh muscles of mice treated with or without the Piper retrofractum Vahl. Extract of the present invention.

FIG. 9 is a graph showing thigh muscle volumes of mice treated with or without the Piper retrofractum Vahl. extract of the present invention. The marks * and ** on the bar graph represent significance at *p<0.05 and **p<0.01), respectively, as compared by one-way ANOVA and Tukey's Multiple Comparison Test.

FIG. 10 shows a reduction in intramuscular triglyceride (IMTG) levels as IMTG is used as an energy source in mice treated with Piper retrofractum Vahl. extract of the present invention.

FIG. 11 is a graph showing the positive effect of the Piper retrofractum Vahl. extract of the present invention on exercise performance in mice, as measured on a treadmill. The marks * and ** on the bar graph represent significance at *p<0.05 and **p<0.01), respectively, as compared by one-way ANOVA and Tukey's Multiple Comparison Test.

FIG. 12 shows the activation of pAMPK, pACC, and CPT-1 in mice treated with the Piper retrofractum Vahl. extract of the present invention, as analyzed by Western blotting.

### BEST MODE

A composition comprising a Piper retrofractum Vahl. extract in accordance with the present invention is prophylactic and therapeutic of obesity by decreasing body weight and fat, and of diabetes by reducing blood glucose and insulin levels, and can enhance muscle mass and exercise performance by controlling genes in such a manner as to facilitate intramuscular energy sources (AMPK activation, ACC suppression, and CPT-1 activation).

In one embodiment of the present invention, the Piper retrofractum Vahl. fruit extract is obtained as an organic solvent extract, or as a supercritical extract solution using carbon dioxide alone or in combination with an organic solvent.

### EXAMPLE

### Extraction

Dried powder of Piper retrofractum Vahl. fruits was placed, together with 4 to 10 volumes of an extraction solvent, in an extraction device, and left for 12 hrs or longer or subjected to supercritical extraction, followed by concentration and drying in a concentrator to give an extract.

Examples of the extraction solvent available in the present invention include purified water, methanol, ethanol, propanol, isopropanol, butanol, acetone, ether, benzene, chloroform, ethyl acetate, methylene chloride, hexane, cyclohexane, petroleum ether, and a mixture thereof, with preference for ethanol. However, so long as it elicits an extract from Piper retrofractum Vahl. fruits, any solvent may be used without limitations.

Also, the present invention addresses a functional health food and a pharmaceutical composition, both configured to exhibit anti-obesity and anti-diabetic activity. Containing the Piper retrofractum Vahl. fruit extract of the present invention, the functional health food and the pharmaceutical composition functions to prevent and treat not only obesity by causing the loss of body weight and fat through a UCP activation mechanism in adipose tissue, but also diabetes by improving insulin resistance through the reduction of blood glucose and insulin levels. In addition, the functional health food and the pharmaceutical composition causes fats, an energy source for aerobic exercise, to translocate into muscular mitochondria through a mechanism responsible for AMPK activation, ACC suppression and CPT-1 upregulation to increase muscle mass and enhance exercise performance and capacity, thus bringing about an exercise-mimicing effect.

For use in oral dosage forms, the anti-obesity functional food composition or pharmaceutical composition of the present invention may be formulated, in combination with a suitable carrier, into powders, granules, pills, sugar-coated tablets, tablets, liquids, gels, syrups, suspensions, wafers, etc. Examples of the carriers suitable for oral formulations include saccharides such as lactose, dextrose, sucrose, sorbitol, mannitol, xylitol, erythritol, and maltitol, starches such as maize starch, wheat starch, rice starch and potato starch, celluloses such as cellulose, methyl cellulose, sodium carboxylmethylcellulose, and hydroxyl propylmethyl cellulose, and fillers such as gelatin and polyvinylpyrrolidone. As needed, a disintegrant, such as crosslinked polyvinylpyrrolidone, agar, alginic acid, or sodium alginate may be additionally added. Further, the pharmaceutical composition may further comprise an anticoagulant, a lubricant, a humectants, a flavor, an emulsifier, and a preservative.

### MODE FOR INVENTION

A better understanding of the present invention may be obtained through the following examples which are set forth to illustrate, but are not to be construed as limiting the present invention.

### Experimental Example 1

### Change of Body Weight and Fat in Animal Model

<1-1> Experimental Animals and Foods

In an anti-obesity and anti-diabetes experiment, a high-fat diet with 40% fat calorie was employed while a Piper retrofractum Vahl. extract was fed in an amount of 100 mg per kg of mouse (EXT 100). Male C57BL/6J mice, 5 weeks old, were randomly divided into a high-fat diet control group (HFD group), and test groups (EXT groups) to which the extract was to be administered at different doses, and then allowed to acclimate for one week to a new laboratory environment. They were fed with the high fat diet for 7 weeks, followed by forcibly administering a vehicle or a dispersion of the extract in the vehicle for 8 weeks.

In an experiment for enhancing exercise performance, a high-fat diet with 40% fat calorie was employed while a Piper retrofractum Vahl. extract was fed in an amount of 100 mg (PRE100) or 300 mg (PRE300) per kg of mouse. Male C57BL/6J mice, 5 weeks old, were randomly divided into a high-fat diet control group (HFD group), and two test groups to which the extract was to be administered at different doses (PRE100 and PRE300 groups), and then allowed to acclimate for one week to a new laboratory environment. They were fed with the high fat diet for 7 weeks, followed by forcibly administering a vehicle or a dispersion of the extract in the vehicle for 8 weeks.

<1-2> Measurement of Body Weight and Fat

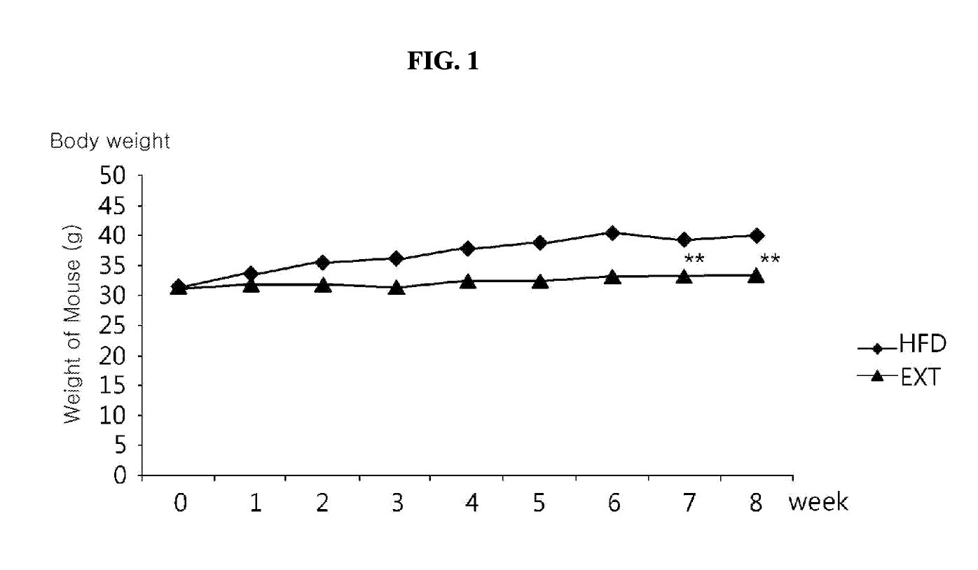

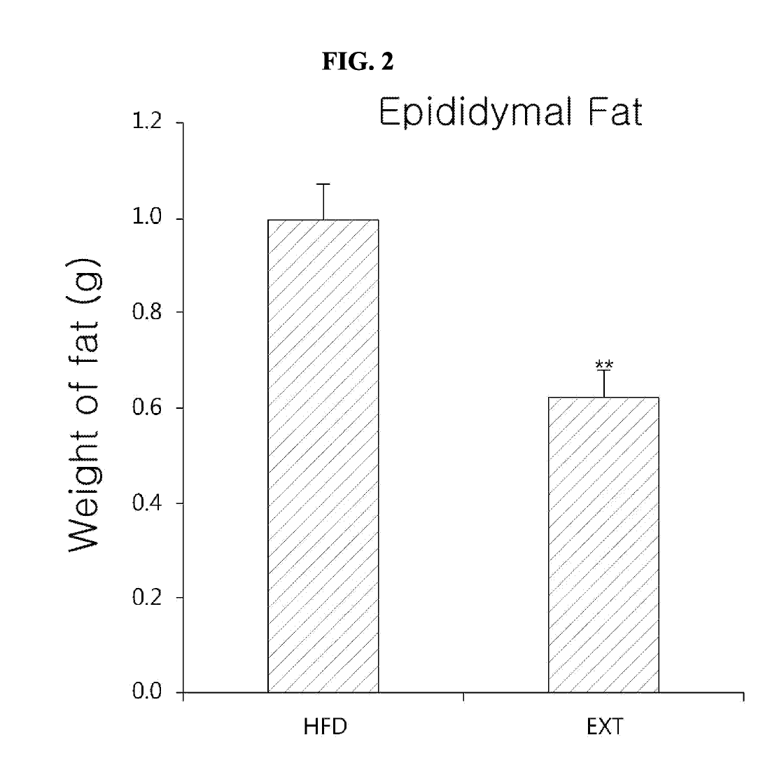

The mice which were forcibly administered with the vehicle or the extract dispersed in the vehicle for 8 weeks were weighed immediately before sacrifice. Then, epididymal fat was excised from the mice and weighed [FIGS. 1 and 2].

### Experimental Example 2

### Blood Glucose, Blood Insulin and HOMA-IR in Animal Model

<2-1> Measurement of Blood Glucose and Insulin

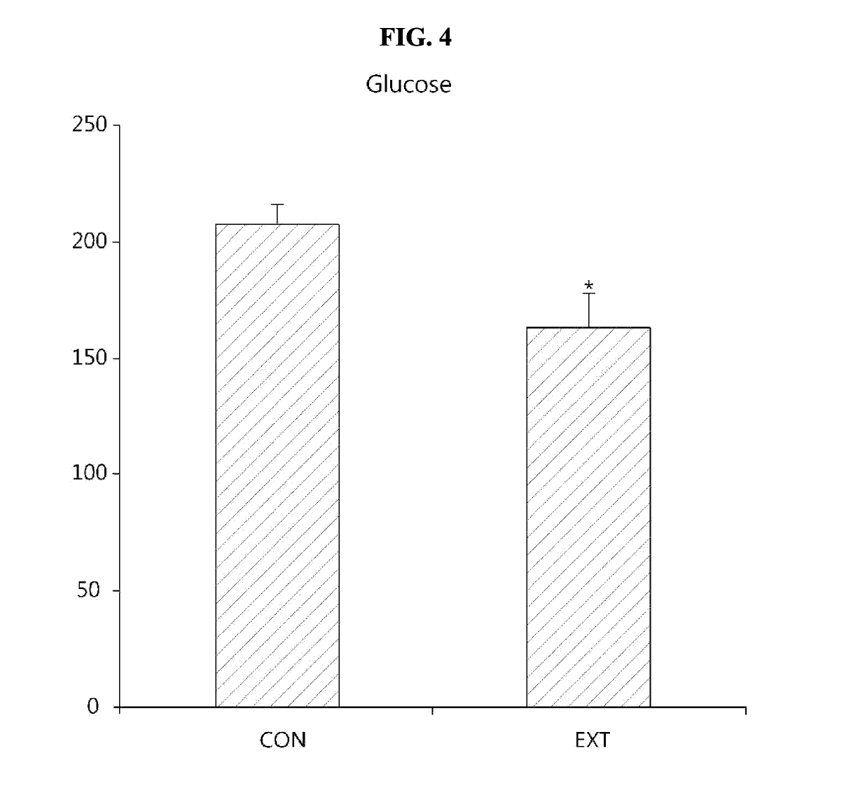

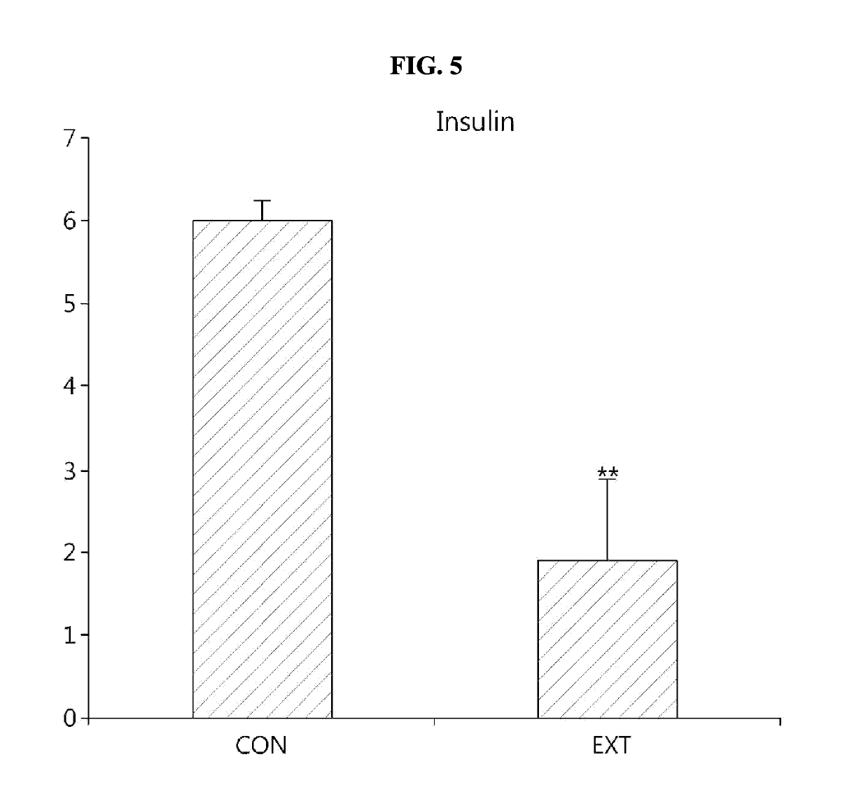

After 8-week uptake of the Piper retrofractum Vahl. extract of the present invention, the mice were fasted for 6 hrs, and sera were taken from the tail and examined for glucose and insulin levels. As shown in FIGS. 4 and 5, blood glucose and insulin levels were significantly decreased in EXT100 group fed with the test material Piper retrofractum Vahl. extract, compared to the control (HFD).

<2-2> Measurement of HOMA-IR

HOMA-IR was calculated from the blood glucose and insulin levels measured in <2-1> according to the following formula.

HOMA-IR=FBG (mg.dL)×FI (ng/ml)×0.0717225161669606

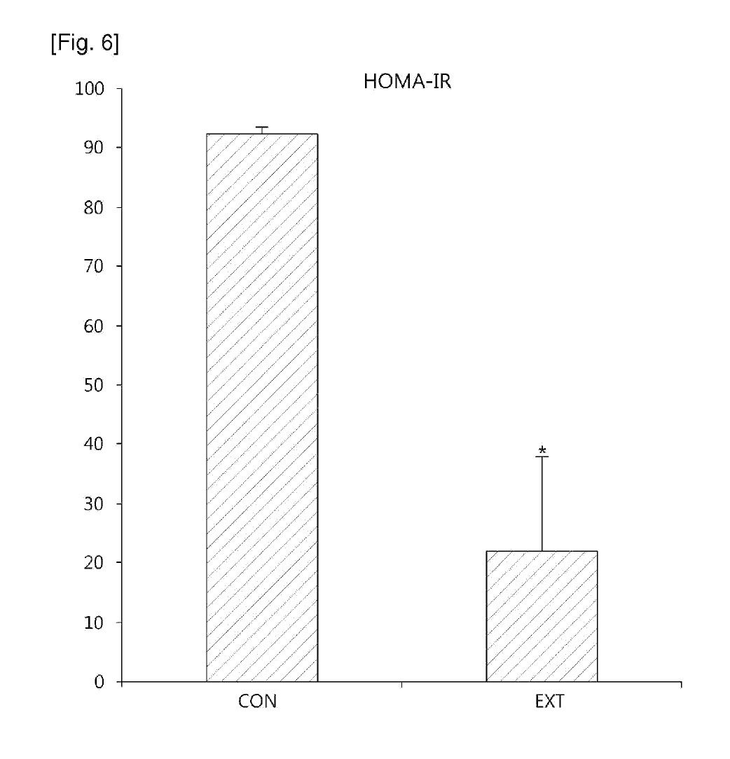

As can be seen in FIG. 6, a significantly lowered HOMA-IR value was detected from the EXT100 group, which was fed with the Piper retrofractum Vahl. extract, compared to the control (HFD), indicating that the extract can improve insulin resistance.

### Experimental Example 3

### Muscle Volume, Muscular Fat, and Exercise Performance in Animal Model

<3-1> Measurement of Muscle Volume

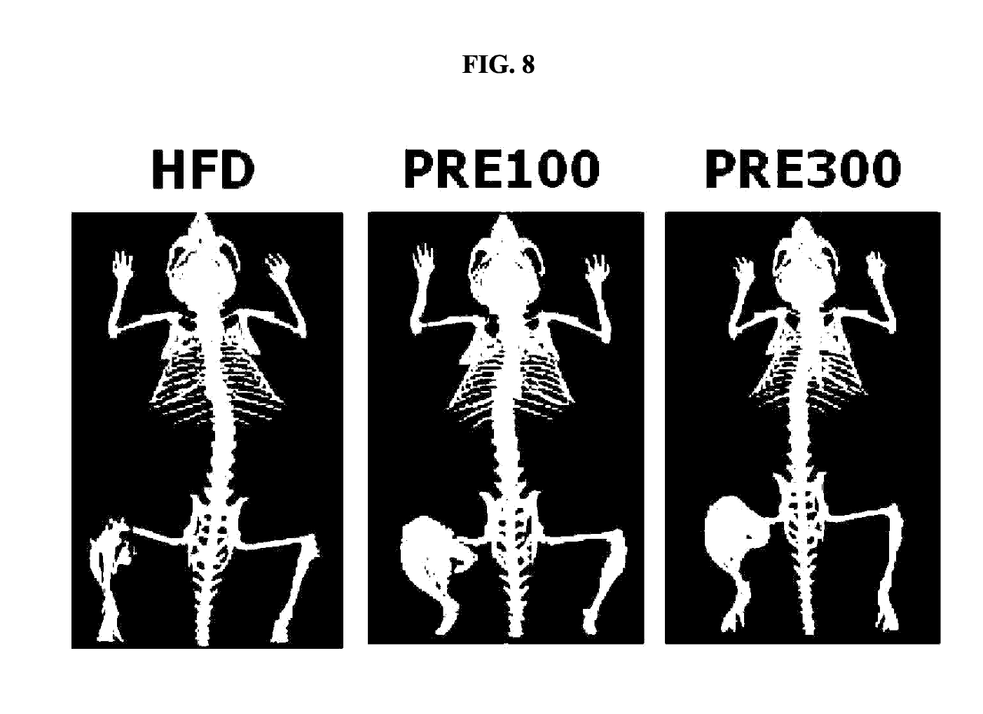

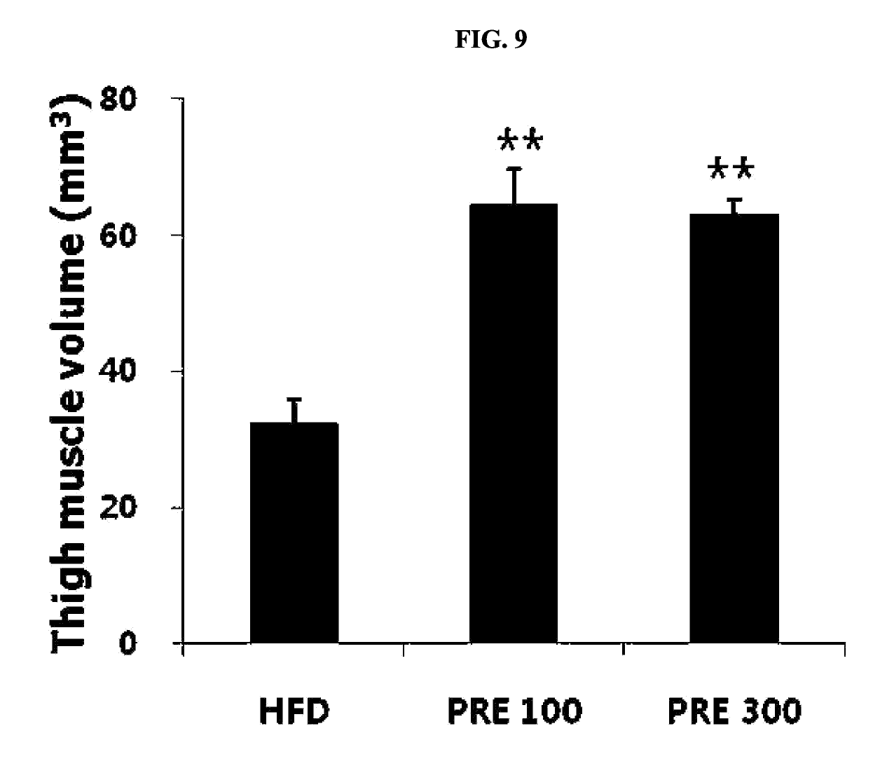

After 8-week uptake of the Piper retrofractum Vahl. extract, the mice were analyzed for muscle volume by micro PET/CT (positron emission tomography/Computed tomography, INVEON, Siemens, USA). As can be seen in FIGS. 8 and 9, PRE100 and PRE300, both fed with the Piper retrofractum Vahl. extracts, were 200% larger in muscle volume, compared to the control group (HFD).

<3-2> Measurement of Muscular Fat

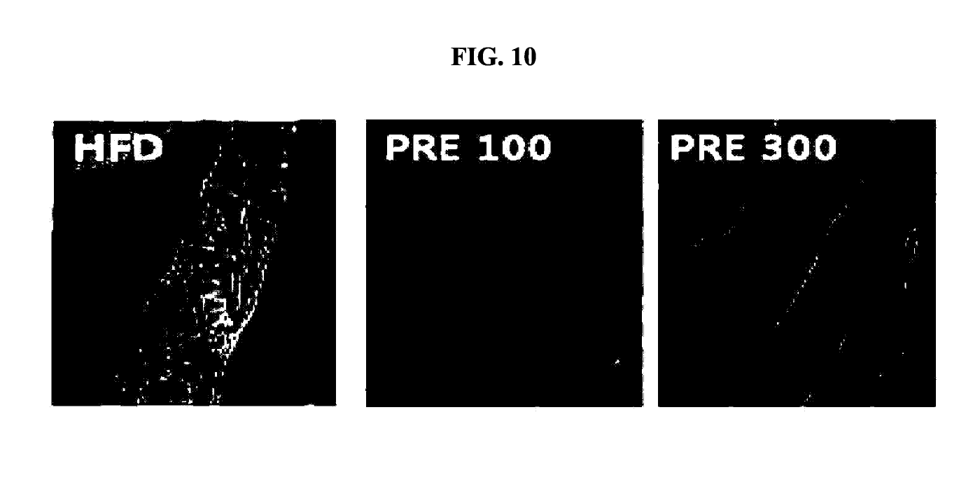

Muscle was exercised from the mice which were sacrificed after 8-week uptake of the Piper retrofractum Vahl. extracts, and stained with hematoxylin and eosin (H&E) to visualize lipids. As can be seen in FIG. 10, a significant reduction in muscular fat content was found in PRE100 and PRE300, both fed with the extract, compared to the control (HFD), indicating that IMTG(intra-muscular triglyceride) stored in muscle was reduced with the production of energy.

<3-3> Test for Exercise Performance

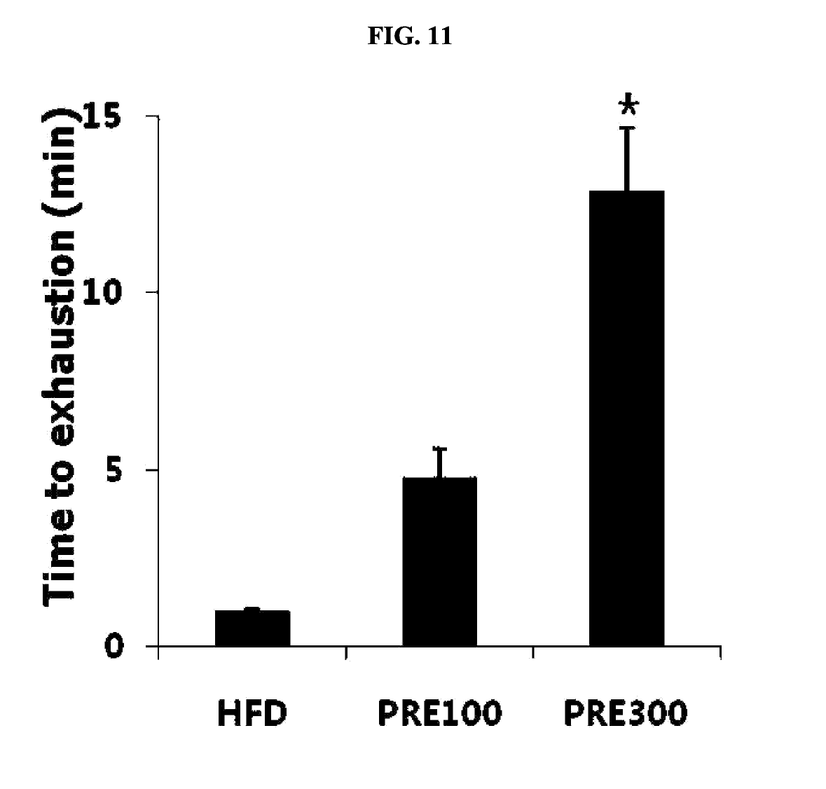

After 8-week uptake of the Piper retrofractum Vahl. extract, the mice were assessed for exercise performance on a treadmill at a speed of 11 m/min. As can be seen in FIG. 4, exercise performance was significantly increased in PRE100 and PRE300, both groups fed with the extract, compared to the control (HFD) (FIG. 11).

### Experimental Example 4

### Upregulation of Genes Responsible for Anti-Obesity, Anti-Diabetes, and Exercise Performance Enhancement in Animal Model

<4-1> Effect of Thermogenesis on Expression of Anti-Obesity-Related Gene

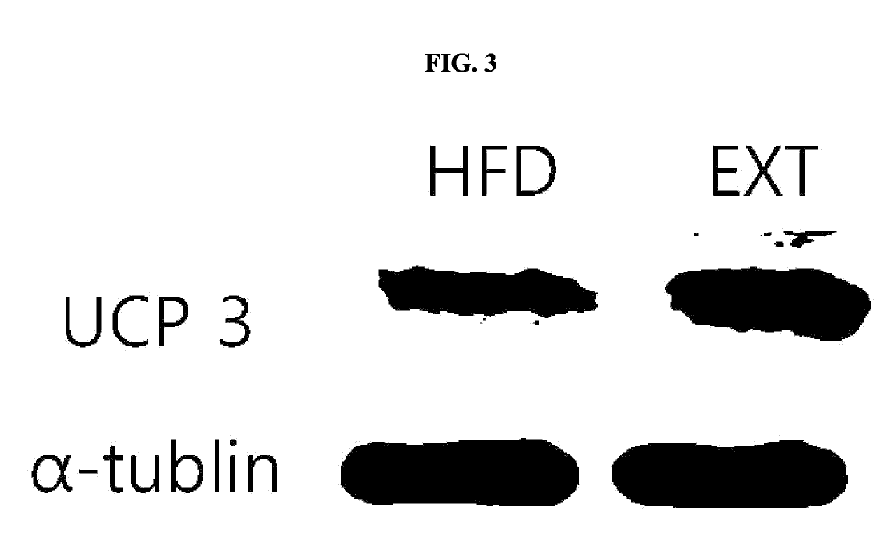

3T3-L1 adipose cells were incubated for 10 days with insulin, dexamethasone, and IBMX to form fat, and then with the Piper retrofractum Vahl. extract. Western blotting showed the expression of UCP proteins [FIG. 3], with a-tublin spots indicating constant loads of samples.

<4-2> Effect on Expression of Anti-Diabetes-Related Gene

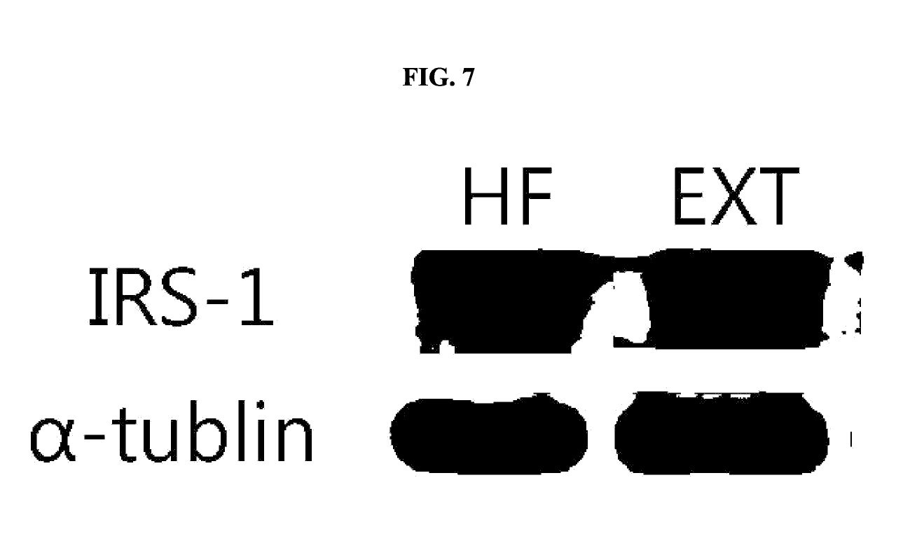

The liver was excised from mice which had been fed for 8 weeks with the Piper retrofractum Vahl. fruit extract, and subjected to Western blotting. The blots show that the extract upregulates the expression of IRS-1 [FIG. 7], with α-tublin blots indicating constant loads of the samples.

<4-3> Effect on Expression of Gene Relevant to Exercise Performance

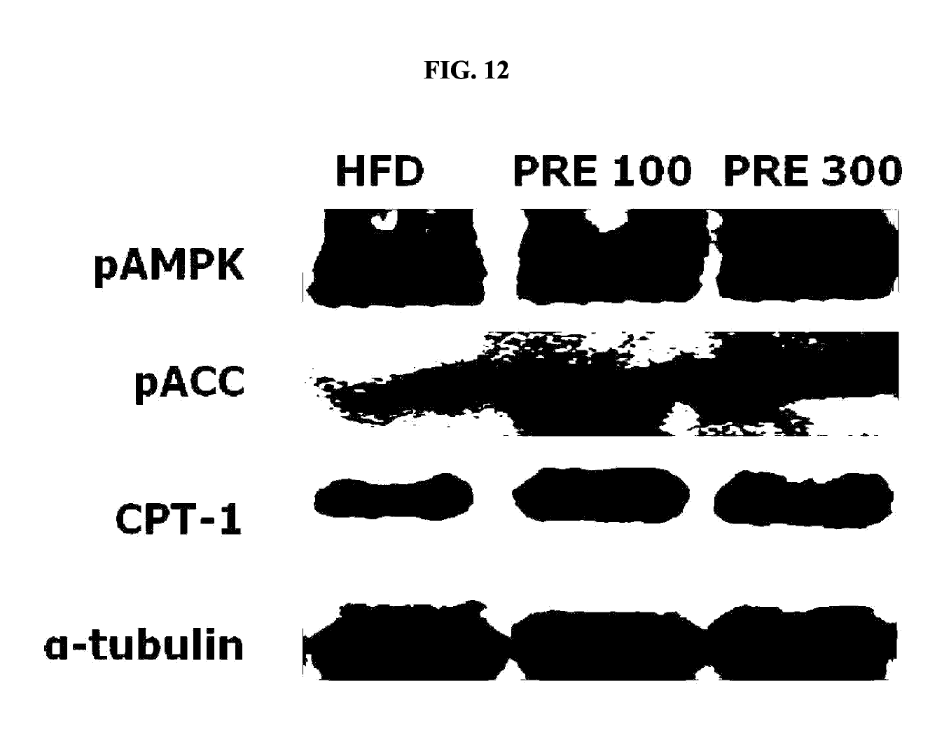

Thigh muscles were excised from mice which had been fed for 8 weeks with the Piper retrofractum Vahl. fruit extract, and subjected to Western blotting. Western blots showed the expression of pAMPK, pACC, and CPT-1 [FIG. 12], with α-tublin blots indicating constant loads of the samples. The extract was observed to upregulate the expression of pAMPK, pACC, and CPT-1.

### INDUSTRIAL APPLICABILITY

As elucidated hitherto, the present invention provides an anti-obesity functional composition derived from a Retrolactum fruit extract. Particularly, the present invention provides a functional composition effective for decreasing body weight and fat on the basis of UCP activation-induced thermogenesis, an anti-diabetic functional composition for the improvement of insulin resistance by reducing blood glucose and insulin levels, and an anti-diabetic functional food composition or pharmaceutical composition, characterized by IRS-1 activation. In addition, the present invention addresses a food or pharmaceutical composition, characterized by AMPK activation-driven ACC suppression and CPT-1 activation, for increasing muscle mass, enhancing exercise capacity and performance, and overcoming fatigue. Consequently, the present invention is very useful in terms of health and medicine.

1. An anti-obese functional food composition, comprising a Piper retrofractum Vahl. fruit extract as an active ingredient, wherein the active ingredient triggers a mechanism responsible for loss of body weight and fat.

2. The anti-obese functional food composition of claim 1, wherein the Piper retrofractum Vahl. fruit extract is obtained with an organic solvent or by supercritical extraction.

3. An anti-obese pharmaceutical composition, comprising a Piper retrofractum Vahl. fruit extract as an active ingredient, wherein the active ingredient triggers a UCP activation-induced thermogenesis mechanism.

4. The anti-obesity quasi-drug product of claim 3, wherein the composition exhibits a mechanism for body weight and fat loss.

5. The anti-obesity pharmaceutical composition of claim 3, wherein the Piper retrofractum Vahl. fruit extract is contained in an amount of 0.001 to 80 wt %, based on the total weight of the composition.

6. An anti-obesity functional food composition, comprising the composition of claim 1, wherein the composition of claim 1 is in a form of a powder, a granule, a tablet, a capsule, a syrup or a beverage, and evokes UCP activation-induced thermogenesis to cause a loss of body weight and fat.

7. An anti-diabetic functional food composition for improving insulin resistance, comprising a Piper retrofractum Vahl. fruit extract as an active ingredient, wherein the active ingredient decreases a blood glucose level and a blood insulin level, simultaneously.

8. The anti-diabetic functional food composition of claim 7, wherein the Piper retrofractum Vahl. fruit extract is obtained from a fruit powder with an organic solvent or by supercritical extraction.

9. An anti-diabetic pharmaceutical composition for improving insulin resistance, comprising a Piper retrofractum Vahl. fruit extract as an active ingredient, wherein the active ingredient trigger an IRS-1 activation mechanism.

10. The anti-obesity quasi-drug product for improving insulin resistance according to claim 9, wherein the composition exhibits a mechanism of reducing blood glucose and blood insulin levels.

11. The anti-obesity pharmaceutical composition of claim 3, wherein the Piper retrofractum Vahl. fruit extract is contained in an amount of 0.001 to 80 wt %, based on the total weight of the composition, and triggers a mechanism of reducing blood glucose and blood insulin levels.

12. The anti-diabetic functional food composition of claim 7, being in a form of a powder, a granule, a capsule, a syrup, or a beverage.

13. A health functional food composition, comprising a Piper retrofractum Vahl. fruit extract as an active ingredient, wherein the active ingredient stimulates a mechanism of increasing muscle mass, enhancing exercise performance and overcoming fatigue.

14. The health functional food composition of claim 13, wherein the Piper retrofractum Vahl. fruit extract is obtained from a fruit powder with an organic solvent or by supercritical extraction.

15. A pharmaceutical composition, comprising a Piper retrofractum Vahl. fruit extract as an active ingredient, wherein the active ingredient stimulates an AMPK activation mechanism.

16. The pharmaceutical composition of claim 15, wherein the composition functions to increase muscle mass, enhance exercise performance, and overcome fatigue.

17. The pharmaceutical composition of claim 15, wherein the Piper retrofractum Vahl. fruit extract is contained in an amount of 0.001 to 80 wt %, based on the total weight of the composition, and functions to increase muscle mass, enhance exercise performance, and overcome fatigue.

18. The health functional food composition of claim 13, wherein the composition is in a form of a powder, a granule, a tablet, a capsule, a syrup, or a beverage, and functions to increase muscle mass, enhance exercise performance, and overcome fatigue.
